(19) United States
(12) Plant Patent Application Publication
(10) Pub. No.: US 2011/0145957 P1
(43) Pub. Date: Jun. 16, 2011

(54) Apple Tree Named 'New York 2'

(50) ;

(76) Inventors: Susan K. Brown, Geneva, NY (US); Kevin Maloney, Phelps, NY (US)

(21) Appl. No.: 12/653,612

(22) Filed: Dec. 15, 2009

(51) Int. Cl. A01H 5/00

(52) U.S. Cl. PLT/172

(57) ABSTRACT

A new and distinct Malus domestica apple tree cultivar named ‘New York 2’ that is characterized by having red, juicy, crisp fruits with a firm texture and tart flavor that ripen mid to late season. The fruit of ‘New York 2’ has a very long storage life (100-150 days) while maintaining fresh quality. Trees are annually productive, grower friendly and fruits are not subject to pre-harvest drop and have good retention on the tree. ‘New York 2’ was tested as NY 92609-463.

### 1. LATIN NAME OF THE GENUS AND SPECIES OF THE PLANT CLAIMED

Malus domestica

### 2. VARIETY DENOMINATION

New York 2

### 3. BACKGROUND OF THE INVENTION

The present invention relates to a new and distinct variety of apple tree botanically known as Malus domestica ‘New York 2’ and hereinafter referred to by the variety denomination of ‘New York 2’.

‘New York 2’ was selected for its precocious tree habit and excellent fruit characteristics at harvest and after a period in cold storage. ‘New York 2’ fruit are very firm, crisp, juicy, and tart.

The new variety was derived from crossing the apple variety ‘Braeburn’ with the apple variety ‘Autumn Crisp’ in 1992. ‘Braeburn’ is a hybrid of ‘Lady Hamilton’ by an unknown pollen parent. ‘Autumn Crisp’ is a hybrid of ‘Golden Delicious’×‘Monroe’ and ‘Monroe’ is a hybrid of ‘Jonathan’×‘Rome Beauty’. One seedling, designated NY 92609-463, was selected from a population of 1,017 seedlings from this cross. Pollination, cultivation and selection were conducted at the New York State Agricultural Experiment Station in Geneva, N.Y.

NY 92609-463 is being named and released as ‘New York 2’ and is the subject of this invention.

Asexual reproduction by budding of the new cultivar ‘New York 2’ in orchards of Cornell University's New York State Agricultural Experiment Station in Geneva, N.Y. by the inventors shows that the unique combination of characteristics of asexually propagated trees are true to form and transmitted through succeeding propagations.

### 4. BRIEF SUMMARY OF THE INVENTION

The following traits of the new cultivar ‘New York 2’ have been observed and documented in multiple years. The observations and description were collected from test trees grown in Geneva, N.Y. and other test sites within New York State.

The new variety is distinguishable from the parent varieties ‘Braeburn’ and ‘Autumn Crisp.’ Multiple features distinguish the new variety from the parent varieties.

‘Braeburn’ fruit have orange-red fruit color, slightly oblique fruit shape, and later maturity when compared to ‘New York 2.’ ‘Autumn Crisp’ fruit has earlier maturity, red blush color on a yellow/green background, and a globose fruit shape when compared to ‘New York 2.’ ‘New York 2’ has large fruit size and a brighter and deeper and more uniform red color pattern of fruit. Fruit of ‘New York 2’ mature after ‘Autumn Crisp’ and before ‘Braeburn.’

‘New York 2’ has foliage similar to ‘Braeburn,’ is annually productive and the fruits are retained without pre-harvest drop similar to its ‘Autumn Crisp’ parent.

‘New York 2’ has not been grown and observed in all geographic locations and all possible climatic conditions. Slight phenotypic variations might be observed over locations without any change to the genotype.

### 5. BRIEF DESCRIPTION OF THE DRAWINGS

The accompanying color photographs illustrate typical specimens of the foliage and fruit and typical anatomical characteristics of the new Malus variety ‘New York 2,’ showing the colors as true as is reasonably possible with colored reproductions of this type. Colors in the photographs may differ slightly from the color values cited in the detailed botanical description, which accurately describe the color of ‘New York 2.’

FIG. 1 shows a ‘New York 2’ flower cluster with the king flower open.

FIG. 2 shows 6 different flowers, two flowers each (from left to right) of the variety ‘Braeburn’ (far left), ‘New York 2’ (middle) and ‘Autumn Crisp’ (far right).

FIG. 3 shows six views of representative apples of ‘New York 2.’ Beginning in the upper left corner and proceeding clockwise:

View A is a cross-sectional view of ‘New York 2’ fruit cut longitudinally.

View B is a side view.

View C is a top (stem-end) view.

View D is a bottom (calyx end) view.

View E is a side view.

View F is a cross-sectional view of ‘New York 2’ fruit cut horizontally.

FIG. 4 shows two mature leaves of ‘New York 2,’ one top view and one bottom view.

FIG. 5 is a young tree of ‘New York 2.’

FIG. 6 shows ‘New York 2’ apples on the tree.

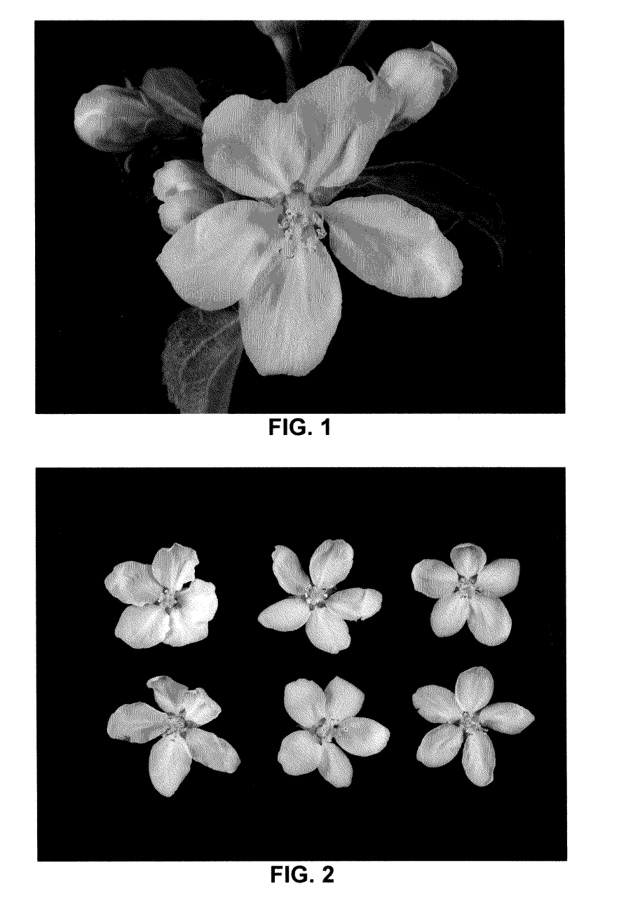

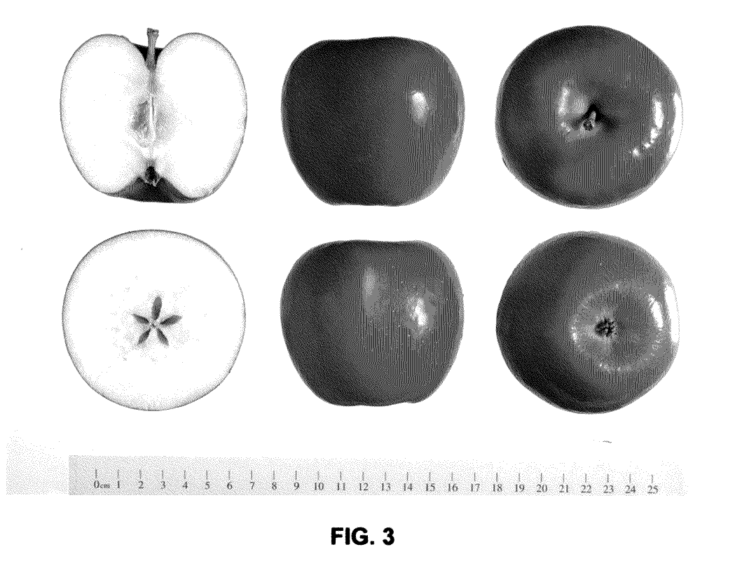

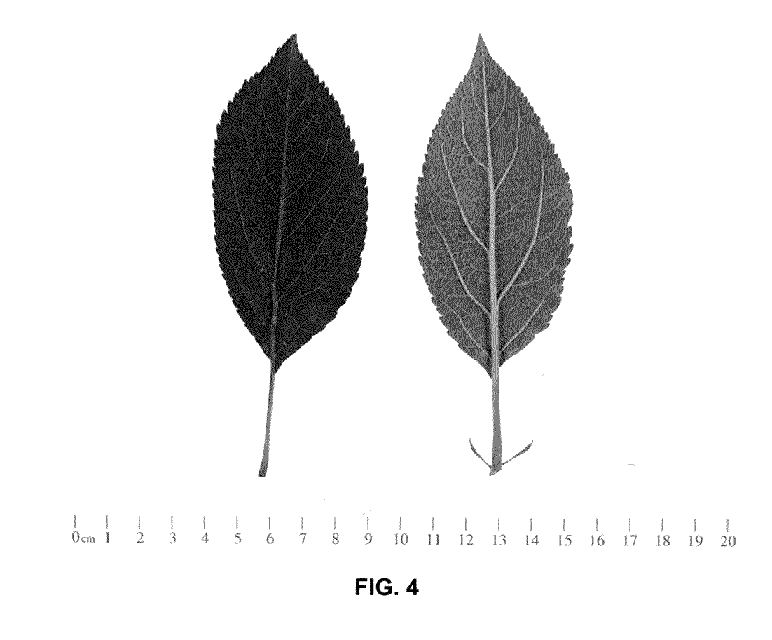

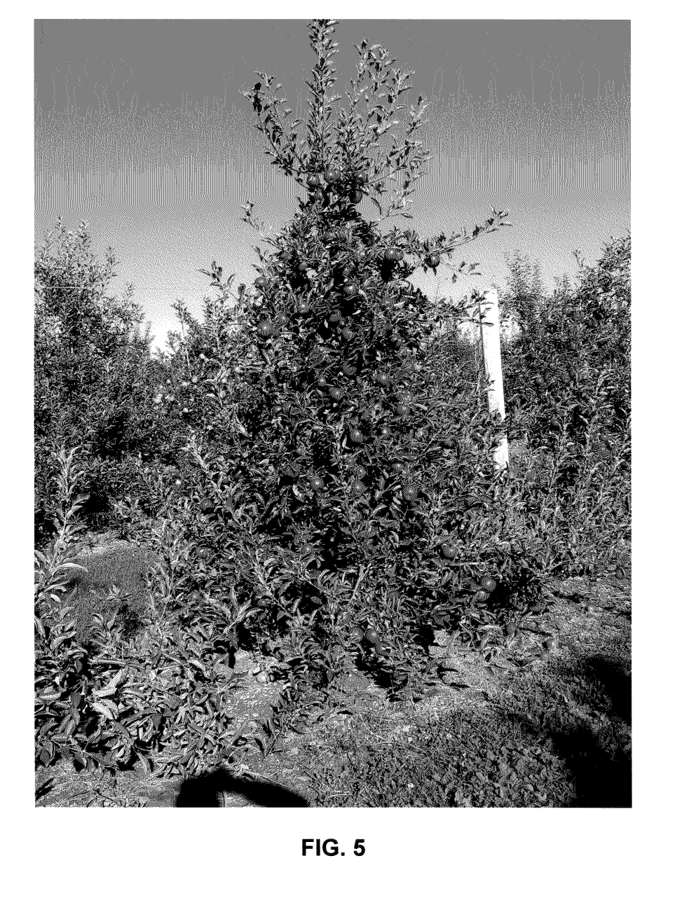

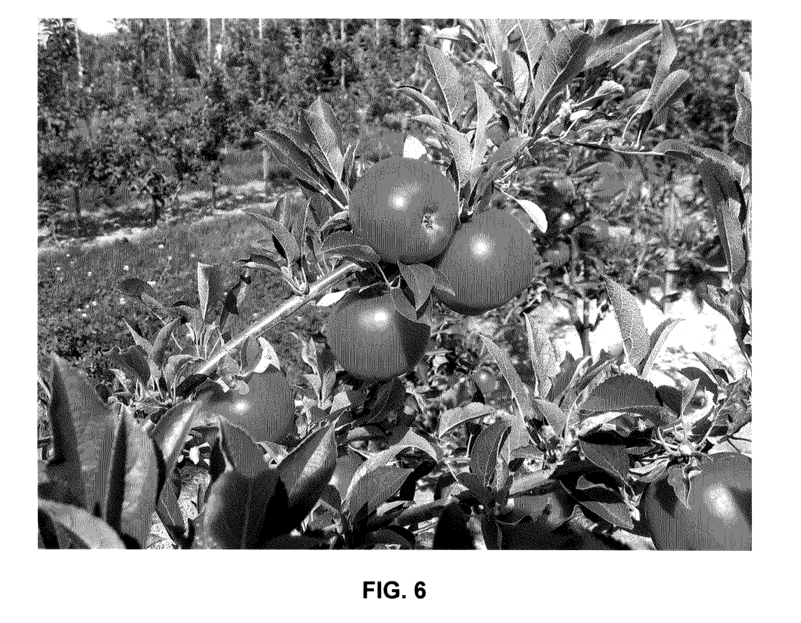

### 6. DETAILED BOTANICAL DESCRIPTION

The following is a detailed botanical description of the new variety with color terminology in accordance with The Royal Horticultural Society Colour Chart (R.H.S.C.C.) except where general color terms of ordinary meaning are used as is clear from the context.

The specimens described were grown at the New York State Agricultural Experiment Station in Geneva, N.Y. in the United States of America. The observations were made on trees grown from 1993 to 2009.

Flowering begins on average by May 9th and full bloom is by May 13th in Geneva, N.Y. The fruit ripen for eating towards mid to late season. Specifically harvest commences on about October 13th and ends about October 28^{th} in Geneva, N.Y.
Trees: medium; spreading habit; early bearing on spurs; vigor medium.
Trunk: Smooth, size, medium; the one-year-old bark is RHSCC (brown group 200B and greyed-purple 187A).
Branches: Moderately thick; smooth; multi-branching; the angle of branching being commonly 30-40 degrees above the horizontal.
Lenticels: Average; small.
Leaves: Medium size, upward pose, concavo-convex in cross section, serrate indentation of margin, medium glossiness of upper sides; medium pubescence on lower side; medium petiole length; medium stipule size; medium time of bud burst. Average leaf blade length 116 mm, blade width 53 mm, petiole length 40 mm and averages 3.8 serrations per cm of leaf blade margin. The color of the upper surface of the leaf blade is most similar to RHCSS 147A, the bottom surface of the leaf blade is most similar to RHSCC 146A, the veins are 145C and the petioles are RHSCC148D.
Flowers: Medium time of beginning of flowering (10% flowers); six flowers per cluster, flowers in cluster overlap, large size (52 mm flower diameter); flat shape; margins of petals slightly overlap; bud color of unopened flower, light pink (RHSCC red group 54A).
Flower petal color: white (RHSCC white group 155B), 5 petals per flower, petal shape is oval, petal length 23 mm, petal width 16 mm.
Flower pedicel color: RHSCC yellow green 148A, length 26 mm, width 1.5 mm.
Stamens: 22 stamens per flower, 9-14 mm long, stamen color RHSCC green white group 157C. Anther color RHSCC greyed yellow 162C.
Stigma: 6 mm long, 5 styles per flower — style color greyed green 195C.
Sepals: Sepal color RHSCC yellow-green group 146B and tinged at tip RHSCC greyed-purple 184A, sepal length 8 mm, sepal width 2 mm, sepals slightly reflexed, nearly flat position, 22 anthers per flower, stigma color RHSCC grey brown 199C.
Fruit:
Fruit: Examined at maturity.
Soluble solids: 13.7.
Acidity: 0.61% malic acid as measured by titration.
Lbs. Pressure: 16 to 21 as measured by penetrometer.
Size: Large to very large; axial diameter (8.2 cm), transverse diameter (7.2 cm), weight 210 grams.
Shape: Uniform; globose; symmetrical in side view; medium crowning at distal end.
Cavity: Obtuse. RHSCC yellow-green 152C and ranging from 152A to 152D. Depth 12.2 mm, width 28.8 mm.
Basin: Wide, broad breadth; wavy. Depth 11.4 mm, width 29.2 mm.
Stem: Medium thickness 2.0 mm; medium length 15.6 mm.
Calyx: Persistent, partially closed, erect.
Calyx tube: Cone shaped.
Sepal: Length, medium; spacing, loosely closed not touching.
Stamen remnants: Median.
Core lines: Meeting.
Core position: Median, closed core, medium core (24.3 mm in width).
Carpels: Ovate, inner surface of carpel slightly tufted.
Skin: Medium-thick; smooth; bloom of skin, scant; cracking tendency of skin, absent; background color, RHSCC greyed-yellow 160B.
Over-color: Approximately eighty percent of over-color of skin; red, RHSCC greyed purple 185A mostly solid blush with small dots.
Flesh: Juicy, crisp, firm: penetrometer (16-21 lbs. at harvest).
Texture: Crisp, slightly coarse, firm.
Flavor: Brix 13.7; acidity 0.61 tart and pleasant mild flavor.
Weight of fruit: 210 gm.
Quality: Very good, fresh, juicy, firm and crisp.
Seeds: Five locules; 9 to 11 seeds total, seed length 10.4 mm, seed width 4 8 mm, seed depth 2.7 mm, acuminate and acute, tufted, dried color RHSCC 165A.
Use: Market; dessert, fresh slice, multiple.
Keeping quality: Excellent: no disorders after 100 days.
Resistance to insects: Good.
Resistance to diseases: Good, but moderately susceptible to powdery mildew (Podosphaera leucotricha) of the foliage.
Production: Early, regular cropping and very precocious.
Growth habit: Standard, fruit borne on short spurs.
Management: Trees require pruning in winter and fruit thinning in early summer Trees in test plot trained as center leader or slender spindle trees. Natural habit is medium vigor, rounded crown (fruiting leader), with many branches. ‘New York 2’ has been successfully grown on EMLA 9 and M.9 (337) rootstocks.

1. A new and distinct Malus domestica apple tree variety named ‘New York 2’ as described and illustrated herein.
